(19) United States
(12) Patent Application Publication
(10) Pub. No.: US 2015/0219646 A1
(43) Pub. Date: Aug. 6, 2015

(54) Compositions and Methods for the Detection of Bacterial Infections Associated with Lyme Disease

(71) Applicant: The General Hospital Corporation, Boston, MA (US)

(72) Inventors: John A. Branda, South Grafton, MA (US); Mary Jane Ferraro, Wellesley Hills, MA (US); Allen C. Steere, Wellesley, MA (US)

(73) Assignee: The General Hositpal Corporaion, Boston, MA (US)

(21) Appl. No.: 14/421,820

(22) PCT Filed: Aug. 15, 2013
(86) PCT No.: PCT/US2013/055123

§ 371 Date: Feb. 14, 2015

Related U.S. Application Data
(60) Provisional application No. 61/683,945, filed on Aug. 16, 2012.

(51) Int. Cl. G01N 33/569

(52) U.S. Cl. CPC G01N 33/56911; G01N 2333/20; G01N 2469/20

(57) ABSTRACT

The invention is directed to methods of assaying biological samples for the presence of antibodies or antigens indicative of infection by a bacterium of the Borrelia burgdorferi sensu lato complex. Also included in the invention are devices that can be used in carrying out these methods. The methods and devices may be used to help identify subjects that have Lyme disease.

### FIELD OF THE INVENTION

The present invention is directed to strategies for testing biological samples to determine if they have characteristics suggesting infection by bacteria of the Borrelia burgdorferi sensu lato complex and to devices that can be used in implementing those strategies.

### BACKGROUND OF THE INVENTION

Lyme disease is an infection transmitted by ticks carrying various species of bacteria of the genus Borrelia. Although many species of Borrelia have been associated with the disease, B. burgdorferi sensu stricto, B. afzelii, and B. garinii appear to be the ones most often responsible for human infections (Wang, et al., Clin. Microbiol. Rev. 12:633-653 (1999)). Lyme disease occurs in many mammalian species, including various forms of wildlife, dogs, cats, and humans (Donahue, et al., Am. J. Trop. Med. Hyg. 36:92-96 (1987); Green, et al., J. Clin. Micro. 26:648-653 (1988)).

If left untreated, Lyme disease can cause serious damage to the nervous and musculoskeletal systems of an infected individual. Thus, early diagnosis and treatment are essential. Unfortunately, identifying Lyme-related bacterial infections is difficult, especially during the early stages of the disease (see generally Maloney, J. Am. Phys. and Surgeons 14:82-89 (2009)).

Serologic testing remains the most useful laboratory tool to support the diagnosis of Lyme disease. Currently, the Centers for Disease Control and Prevention (CDC) endorses a 2-tiered serodiagnostic algorithm, in which the first test is a sensitive enzyme immunoassay (EIA) or immunofluorescent assay (IFA) (Morb. Mortal. Wkly. Rep. 44(31):590-591(1995)). Typically, U.S. laboratories choose a polyvalent, whole-cell sonicate (WCS) EIA as a first-tier test. Specimens that are positive or equivocal by the first test are analyzed using IgM and IgG WCS Western blots, which must be interpreted using specified criteria (Morb. Mortal. Wkly. Rep. 44(31):590-591(1995)).

One major drawback of this approach is its reliance on Western immunoblotting. The difficulty of Western blotting has led to inter-laboratory and intra-laboratory variation (Binnicker, et al., J. Clin Microbiol 46(7):2216-21 (2008)) and to false-positive results caused by over-interpretation, a particular problem with IgM blots (Aguero-Rosenfeld, et al., Clin. Microbial. Rev. 18(3):484-509 (2005)). Most clinical laboratories in the United States do not attempt this procedure themselves and instead rely on commercial reference laboratories, increasing both cost and turnaround time. In a recent proficiency survey administered by the College of American Pathologists, only 54 of 330 participating laboratories (16%) performed LD Western blots in-house (College of American Pathologists, Tick-transmitted diseases survey TTD-B (2010)).

A number of reports have shown that an EIA detecting antibodies directed against the variable-major protein-like sequence, expressed (VlsE) lipoprotein of B. burgdorferi or against a 26-mer peptide from the 6^{th} invariable region of the protein performs similarly to standard 2-tiered testing (Lawrenz, et al., J. Clin. Microbial. 37(12):3997-4004 (1999); Bacon, et al., J. Infect. Dis. 187(8):1187-99 (2003); Liang, et al., J. Clin. Microbial. 37(12):3990-6 (1999); Mogilyansky, et al., Clin. Diagn. Lab. Immunol. 11(5):924-9 (2004)). These findings raise the possibility that the 2-tiered approach may be replaced with a single, objective, and easier to perform EIA. However, the results from single-test approaches using a C6 EIA (Steere, et al., Clin. Infect. Dis. 47(2):188-95 (2008); Ledue, et al., Clin. Vaccine Immunol. 15(12):1796-804 (2008)), or a recombinant VlsE immunoassay (Ledue, et al., Clin. Vaccine Immunol. 15(12):1796-804 (2008)), or a recombinant VlsE immunoblot (Branda, et al., Clin. Infect. Dis. 50(1):20-6 (2010)), suggest that these approaches are not as specific as the 2-tiered approach.

A report was recently published on a modified 2-tiered algorithm that incorporated VlsE testing (Branda, et al., Clin. Infect. Dis. 50(1):20-6 (2010)). The first-tier test remained a polyvalent WCS EIA, while the second-tier test was an IgG Western blot, modified by the addition of a recombinant VlsE band. IgM testing was not performed in the second tier. A key finding was that none of 252 control sera was positive by both WCS EIA and the VlsE band, whereas either test alone produced false-positive results (Branda, et al., Clin. Infect. Dis. 50(1):20-6 (2010)). Since VlsE expression in B. burgdorferi is lost or reduced after repeated in-vitro passage (Norris, et al., Infect. Immun. 60(11):4662-72 (1992); Zhang, et al., Cell 89 (2):275-85 (1997)), spirochetal lysates used to manufacture WCS EIAs contain little VlsE, which probably explains this finding. The same study also showed that a 2-tiered algorithm consisting only of WCS EIA followed by VlsE testing on an IgG immunoblot provided comparable sensitivity as standard 2-tiered testing (Branda, et al., Clin. Infect. Dis. 50(1):20-6 (2010)). Although this modified algorithm removed one of the main drawbacks of standard 2-tiered testing (the IgM Western blot), the second-tier test (VISE band) still relied on immunoblotting.

In response to correspondence (Porwancher, J. Infect. Dis. 189(10):1962 (2004)), Bacon et al. reanalyzed data from a previous study and showed that a WCS ELISA followed by C6 IgG and pepC10 IgM ELISAs would have performed comparably to standard 2-tiered testing (Johnson, et al., J. Infect. Dis. 189(10):1962-4 (2004)). Janssen et al. designed a similar strategy for use in Europe using 2 IgG ELISAs, one with multiple recombinant antigens and one with the C6 peptide. This approach in patients with stage 2 or 3 LD showed comparable sensitivity and better specificity than 2-tiered testing using European IgG EIA and immunoblot assays (Jansson, et al., Clin. Microbiol. Infect. 11(2):147-50 (2005); Nyman, et al., Clin. Microbiol. Infect. 12(5):496-7 (2006)).

### SUMMARY OF THE INVENTION

The present invention is directed to methods that can be used to help determine if a subject has been infected with bacteria associated with Lyme disease. In addition, the invention encompasses certain devices that can be used in carrying out these methods.

In its first aspect, the invention is directed to a method of diagnostically evaluating a subject for infection by Borrelia burgdorferi sensu lato. The method involves obtaining a fluid test biological sample from the subject and, in a first step, assaying this sample for antibodies recognizing a whole-cell lysate of the bacteria. Although any type of assay could be used for this analysis, preferred assays are enzyme immunoassays (EIAs), such as the commercially available EIAs marketed by VIDAS® (bioMérieux SA; Marcy-l'Etoile, France) or Alere® Inc. (Waltham, Mass.). For the purposes of the present invention, an antibody “recognizes” antigens if it binds more strongly to those antigens than other antigens present in a given sample. For example, antibodies in a blood sample that recognize antigens of bacteria of the genus Borrelia would bind to a greater extent to these antigens than to antigens from bacteria in a different genus, preferably in a ratio of more than 10 to 1 and more preferably 100 or 1000 to 1. An alternative preferred assay is a lateral flow immunochromatographic assay similar to the rapid assays marketed by Alere® Inc. (Waltham, Mass.). Other types of immunoassays may also be used but, due to their relative difficulty, assays involving Western blotting should be avoided. The assays may be used to detect either a combination of IgG, IgM and IgA antibodies or may be limited to one specific isotype. The advantage of examining individual antibody isotypes is that, since the level of IgM and IgA antibodies tends to peak sooner after infection than IgG antibodies, the relative type of antibody present may provide a general indication of how recently an infection may have occurred.

The second step in the method involves assaying the test biological sample for antibodies that specifically recognize epitopes of the variable major protein-like sequence, expressed (VlsE) lipoprotein of Borrelia burgdorferi sensu lato. This may be done using the protein itself (either purified or recombinant) or a synthetic peptide corresponding to a portion of the protein. The most preferred assay tests for antibodies binding to the C6 peptide (a 26 amino acid peptide corresponding to the 6^{th} invariable region of the VlsE lipoprotein). Commercially available C6 assays are presently marketed by Idexx Laboratories, Inc., Westbrook, Me. (the “Lyme Quant C₆® assay” and the “Snap4Dx” assay) and by Immunetics, Inc. Boston, Mass. (the “C₆ Lyme ELISA”). This assay may be run either sequentially or concurrently with the assay recognizing whole-cell antigens described above.

Because of their association with Lyme disease, the most preferred bacteria to employ in the above method are from the Borrelia burgdorferi sensu lato complex, especially Borrelia burgdorferi sensu stricto, Borrelia afzelii and Borrelia garinii. Samples, preferably whole blood, plasma or serum, derived from a subject, preferably a human, may be tested for antibodies indicative of infection by these bacteria to help in determining if the subject has, or may develop, Lyme disease. This will be particularly important in instances where a patient is exhibiting symptoms associated with Lyme disease such as fever, skin rash, joint pain or swelling, facial paralysis or headaches, but a diagnosis based solely on clinical impression is uncertain. Other types of fluids that may be used in the method include urine, saliva, cerebrospinal fluid and synovial fluid.

The second assay in the method described above, i.e., the assay for antibodies recognizing VlsE, may be combined with or replaced by assays for antibodies recognizing other antigens characteristic of Borrelia burgdorferi sensu lato. For the purposes of the present invention, an antigen is characteristic of a bacterium if it can be used to distinguish the bacterium from other types of bacteria. For example, an antigen characteristic of Borrelia burgdorferi sensu lato would be present exclusively or, at least to a much greater degree, in Borrelia burgdorferi sensu lato complex species than in other bacterial species. Besides the VlsE lipoprotein and C6 peptide, other antigens that have been described in the art and that may be useful in the present methods include the outer surface protein C (OspC) and a synthetic peptide comprising the C-terminal 10-amino-acid residues of OspC (pepC 10). The presence of antibodies recognizing antigens in a whole cell lysate of B. burgdorferi sensu lato bacteria, together with antibodies recognizing an antigen characteristic of B. burgdorferi sensu lato complex species is an indication that bacterial infection has occurred and is an indication that a subject has or may develop Lyme disease.

In another aspect, the invention is directed to a method of diagnostically evaluating a subject for infection by B. burgdorferi sensu lato in which an assay of biological samples for antibodies recognizing a whole cell lysate of the bacteria and/or antibodies recognizing a specific antigen characteristic of the bacteria are combined with assays of the biological sample for at least one antigen characteristic of the bacteria. In this case, a conclusion may be drawn that the subject has been infected if assays are positive both for antibodies recognizing bacterial antigens and for at least one antigen characteristic of the bacterium. Essentially the same types of assays discussed above may be used in this method with the main difference being the inclusion of an assay for antigens rather than only antibodies. Although enzyme immunoassays represent one type of preferred assay, lateral flow immunochromatographic methods are also highly preferred and offer special advantages with respect to ease of use and suitability for utilization in a physician's office, a clinical laboratory, or at home. Assays for antibodies recognizing a whole cell lysate and/or specific antigens and for an antigen characteristic of a bacterium may be run on separate immunochromatographic devices or the assays may be combined on a single device. As discussed above, preferred test biological samples are blood, plasma or serum and preferred bacteria are members of the Borrelia burgdorferi sensu lato complex

In another aspect, the invention includes devices (also referred to herein as test strips) for lateral flow immunochromatography that can be used in the assays described above. The devices have a support surface that is coated with an absorbent material and also include one or more test-specific molecules that are localized in distinct areas along the surface. The test-specific molecules are immobilized by attachment to the absorbent material and, in this manner, are prevented from migrating along the surface in response to the flow of fluid during chromatography. Immobilized molecules will include: a) at least one antigen or epitope characteristic of Borrelia burgdorferi sensu lato; and/or b) at least one antibody recognizing an antigen characteristic of a bacterium of the Borrelia burgdorferi sensu lato complex. Specific antigens that may be immobilized include VlsE, the C6 peptide; OspC; and pepC10, with the C6 peptide being most preferred. Antibodies recognizing these or other specific antigens, such as outer surface protein A (OspA), outer surface protein B (OspB), flagellar proteins, or BmpA, or peptides derived from these proteins, may also be immobilized with the antigens and, optionally, with antibodies recognizing whole cell bacterial lysates.

Typically, the immunochromatographic devices of the invention will have an elongated shape defining a direction in which sample and chromatographic fluid flow. The antibodies and/or antigens immobilized along the device surface will form distinct regions where corresponding molecules in test samples will bind. Visualization of bound antigens or antibodies may be accomplished using standard procedures well known in the art and may involve the use of dyes, fluorescent labels, entrained metal complexes, enzyme reactions, etc. Preferred antigens immobilized on devices will be derived from Borrelia burgdorferi sensu lato organisms and preferred antibodies immobilized on the devices will recognize antigens from these bacterial species.

### DETAILED DESCRIPTION OF THE INVENTION

The present invention is based upon the development of methods for determining if a subject has been infected by bacteria of the Borrelia burgdorferi sensu lato complex. The methods use a combination of at least two assays to make an accurate diagnosis. These assays are selected from three options: a) an assay that identifies patients having antibodies that recognize antigens in a whole cell bacterial lysate (see e.g., VIDAS® bioMérieux SA); b) an assay that identifies patients having antibodies that recognize a specific bacterial antigen such as the VslE lipoprotein or the C6 peptide (see e.g., U.S. Pat. No. 6,475,492; and U.S. Pat. No. 6,719,983; see also the Lyme Quant C₆® Assay marketed by Idexx Laboratories, Inc. Westbrook, Me. or the C6 Lyme ELISA marketed by Immunetics, Inc. Boston, Mass.); c) an assay recognizing one or more bacterial antigens or epitopes such as VlsE. Information on the isotype of antibody present in the sample (IgG or IgM) may also be obtained and may help to estimate how recently an infection was acquired (for a review of several immunoassays for the detection of Borrelia burgdorferi IgM and IgG antibodies, see Smismans, et al., Clin. Microbiol. Infect. 12:648-655 (2006)).

One of the limitations of serologic testing for Lyme disease is that a host antibody response takes days to weeks to develop, after initial infection. Therefore, in the early acute phase of infection, there is a window period during which infected patients are seronegative, limiting the sensitivity of serologic testing. A test capable of detecting one or more antigens derived from B. burgdorferi sensu lato in human fluid specimens has the potential to be particularly useful during the early phase of infection in order to diagnose Lyme disease in patients prior to seroconversion. Antigen assays that are known in the art (see e.g., Hyde, et al. J. Clin. Microbiol. 27(1):58-6 (1989); Douglas, et al., Biomaterials 32(4):1157-66 (2011)) may be used to help provide guidance with regard to assays for use in conjunction with the methods described herein.

Preferred assays are enzyme immunoassays, e.g. ELISA type assays, and also immunochromatographic assays. A rapid, lateral flow, immunochromatographic test for Lyme disease in human patients was developed by Chembio Diagnostics (Medford, N.Y.) and distributed by Wampole/Inverness (Gomes-Solecki, et al., Archives Int. Med. 161(16):2015-20 (2001)). This point-of-care test, known as the “PreVue™ B. burgdorferi Antibody Detection Assay,” was manufactured using a mixture of recombinant, chimeric proteins representing key Borrelia epitopes. The assay was designed as a first-step serologic test to substitute for a whole-cell lysate ELISA in two-tiered testing procedures. Although the test gained FDA clearance, it is no longer on the market. Nevertheless, this test provides general guidance with respect to immunochromatographic assays that may be used in the present methods. Additional guidance is readily available in the art (see, e.g., U.S. Pat. No. 4,168,146; U.S. Pat. No. 4,313,734; U.S. Pat. No. 5,569,608; U.S. Pat. No. 5,989,921; U.S. Pat. No. 6,485,982; U.S. Pat. No. 6,008,056; U.S. Pat. No. 7,815,853; U.S. Pat. No. 7,932,093; and U.S. Pat. No. 7,972,872).

In a preferred embodiment, separate lines or spots on an immunochromatograhic test strip are used to indicate the presence of either antigens or antibodies in whole blood, serum, plasma, urine, cerebrospinal fluid, saliva, synovial fluid, or other body fluids. B. burgdorferi sensu lato antigens, including OspA, OspB, OspC, flagellar proteins, BmpA, and VlsE, or peptides derived from these proteins, are captured from human fluid specimens using antibodies raised against them and, separately, human antibodies directed against B. burgdorferi sensu lato are captured using antigen targets derived from the infectious agent. Separate lines or spots on the test strip may contain, for example, whole-cell B. burgdorferi sensu lato lysate or C6 peptide as antigen targets to capture and detect human antibodies directed against the agent of Lyme disease.

The methods and devices described herein will be of use to clinicians in diagnosing patients with Lyme disease and, more generally, to scientists studying infections by Borrelia bacteria. The assays may be performed as part of a screening procedure, e.g., for subjects that live or work in areas where ticks transmitting the agent of Lyme disease are prevalent, or in patients exhibiting symptoms that may be attributable to Lyme disease.

Components needed for performing assays associated with the methods described herein may be combined and sold in the form of a kit. For example a kit may include an immunochromatographic device (e.g., a test strip coated with absorbent material with bands of immobilized bacterial antigens or antibodies that recognize bacterial antigens); and one or more other components such as labeling agents. In addition, kits will typically include instructions describing how the various components can be used in performing assays.

### EXAMPLES

In the present study, a 2-EIA strategy for Lyme disease serodiagnosis in the U.S. is evaluated using a polyvalent whole-cell sonicate (WCS) EIA followed in those with a positive or equivocal result, by a C6 EIA. Both EIAs are FDA-cleared and are used widely in the U.S., but are not typically combined in a 2-test approach.

Patients and Methods

Patient Samples

In phase 1, the 2-EIA algorithm was evaluated using sera from well-characterized patients with LD, or symptomatic control subjects, who had been assessed by LD experts. The Human Investigations Committee at Massachusetts General Hospital (MGH) approved the study. The samples included acute and convalescent sera from 63 patients with culture-confirmed erythema migrans (EM). In addition, one pre-treatment specimen was tested from each of 28 patients with active nervous system, heart or joint involvement of the infection. All patients categorized as having LD met the CDC surveillance criteria for the diagnosis (Wharton, et al., MMWR Recomm. Rep. 39(RR-13):1-43 (1990)). Samples were also included from 54 symptomatic patients referred for potential LD who did not meet criteria, and were diagnosed with another illness. All phase 1 samples were derived from a set collected prospectively between 1999 and 2001, and stored frozen at −80 C. These samples were originally tested by other methods, and those findings have been reported (Steere, et al., Clin. Infect. Dis. 47(2):188-95 (2008); Branda, et al., Clin. Infect. Dis. 50(1):20-6 (2010)).

In Phase 2, the 2-EIA algorithm was evaluated using sera that had been submitted to the MGH Clinical Microbiology Laboratories for LD antibody testing during a one-year period, May 1, 2007 through Apr. 30, 2008. All available sera that had positive or equivocal results by a first-step polyvalent WCS EIA were included in the study (N=467). Based on a review of the 467 patients' medical records, 78 were classified as having active LD based on CDC case definition criteria (Wharton, et al., MMWR Recomm. Rep. 39(RR-13):1-43 (1990)). Additionally, samples were obtained from a total of 1,246 healthy control subjects: 66 were collected during routine well office visits (e.g., blood pressure checks) in highly endemic locations in Connecticut and Rhode Island, 1,080 were collected from healthy blood donors in the Boston area, which draws from endemic regions, and 100 were collected from healthy blood donors in New Zealand, a non-endemic region. The sera from Boston blood donors were collected for the present study. The 66 samples from patients in RI and CT were derived from a set collected prospectively between 1999 and 2001, and the samples from blood donors in New Zealand were collected in 2007; these were originally tested by other methods and the findings have been reported (Steere, et al., Clin. Infect. Dis. 47(2):188-95 (2008); Branda, et al., Clin. Infect. Dis. 50(1):20-6 (2010)).

Serologic Testing

All testing was performed according to the manufacturers' instructions. The serum samples, except those collected from Boston blood donors, were tested using the VIDAS® Lyme IgG/IgM assay (bioMérieux SA; Marcy-l'Etoile, France). The Boston blood donor sera were tested using the Wampole® Borrelia burgdorferi IgG/IgM ELISA II assay (Alere, Inc., Waltham, Mass.). Additionally, all samples were tested with the C6 B. burgdorferi ELISA (Immunetics, Inc.; Boston, Mass.). Western immunoblotting was performed using Borrelia B31 IgM and IgG Virablot® test strips (Viramed Biotech AG; Planegg, Germany). The blots were read with the aid of densitometry; a band was defined as positive if its intensity was X≧90% of the cutoff control band's intensity. Western blots were interpreted using standard CDC criteria (Morb. Mortal Wkly Rep. 44(31):590-1 (1995)). Western blotting had been performed in a previous study on the Phase 1 sera, plus the sera from control subjects in RI, CT and New Zealand; likewise, WCS EIA had been performed on the New Zealand control sera (Clin. Infect. Dis. 50(1):20-6. (2010)), and the results were reported again here.

Statistical Analysis.

Differences between proportions were considered significant if the two-tailed P value was ≦0.05, as determined using Fisher's exact test.

Results

Phase 1: Evaluation of Well-Characterized LD Patients and Control Subjects.

The 2-EIA algorithm's sensitivity was first determined using a sera set from 91 well-characterized patients with various manifestations of LD. Among 63 patients with EM (stage 1 LD), the sensitivity of the 2-EIA algorithm was 37% in the acute phase of the illness, a median of 4 days after disease onset, and 89% during convalescence, 3-4 weeks later at the conclusion of antibiotic therapy (Table 1). In comparison, the percentages were 27% and 57%, respectively, for standard 2-tiered testing. The difference in sensitivity between the 2 algorithms was statistically significant for patients in the convalescent phase of EM (P<0.001), whereas it was not significant for those with acute EM (P=0.34). Among 10 patients with stage 2 LD, the 2-EIA algorithm's sensitivity was 100% compared with 40% for standard 2-tiered testing (P=0.01). Although all 10 patients had positive results by WCS EIA, 6 with a positive IgM immunoblot but a negative IgG immunoblot could not be classified as positive according to CDC criteria because the patients had been ill for longer than 1 month (5-8 weeks) at the time of serum collection. Among the 18 patients with stage 3 LD, the sensitivity of both algorithms was 100%.

The 2-EIA algorithm's specificity was evaluated using a set of control sera from 54 symptomatic patients who were referred to a LD clinic for possible B. burgdorferi infection, but who were determined to have another illness. None was positive by either the 2-EIA algorithm or the standard 2-tiered algorithm (Table 3). In contrast, one of 54 control specimens was positive by the C6-peptide EIA, when considered as a stand-alone test.

Phase 2: Evaluation of Routine Sera from LD Patients and Control Subjects.

During the 1-year study period, 4,520 sera were submitted to the MGH Clinical Microbiology Laboratory for LD testing. Of these, 467 (10%) gave positive or equivocal results in the first-step WCS EIA. Upon review of these patients' medical records, it was determined that 78 of 467 specimens (17%) had been collected from patients with active LD, as determined using CDC clinical criteria. In 51 patients with acute EM, the 2-EIA algorithm's sensitivity was 73%, compared with 61% for standard 2-tiered testing (P=0.29; Table 2). In 16 patients with stage 2 LD, the 2-EIA algorithm was 100% sensitive, whereas standard 2-tiered testing was 94% sensitive (P=1.0). All stage 2 LD samples were positive by WCS EIA and IgM or IgG Western blot, but 1 of the 16 patients had been ill for longer than 1 month and had only a positive IgM blot. Therefore, according to CDC guidelines, this specimen was not classified as positive by standard 2-tiered testing. Finally, among 11 patients with stage 3 LD, both algorithms were 100% sensitive.

The specificity of the 2-EIA algorithm was evaluated using sera from 1,246 healthy subjects. Among the 66 well subjects living in highly endemic regions (CT and RI), the specificity of both the 2-EIA algorithm and the standard 2-tiered approach was 98%, compared with 94% for the C6 EIA alone (P=0.37) (Table 3). The specificity was 99.4% for both 2-step algorithms among 1,080 blood donors living in the Boston area, an endemic region for Lyme disease, compared with 98.5% for the C6 EIA alone (P=0.05). Finally, the specificity was 100% for both 2-step algorithms and for the C6 EIA alone among 100 healthy subjects from New Zealand, a non-endemic region for LD.

Combined Results.

The results from study phases 1 and 2 were similar, and therefore, the data was combined for analysis. Early in the infection (stages 1 and 2 LD), the 2-EIA algorithm's sensitivity was similar to the C6 EIA alone (61% compared with 64%, respectively, P=0.71). Both strategies were more sensitive than standard 2-tiered testing, which provided 48% sensitivity (P=0.03 and 0.008, respectively; Table 4). In late disease (stage 3 LD), all three approaches were 100% sensitive. When sensitivity was calculated using all 169 patients with active LD, irrespective of disease stage, the 2-EIA algorithm's sensitivity was 68% compared with 57% for standard 2-tiered testing (P=0.04). The C6 EIA's overall sensitivity was 70%, higher than either 2-step algorithm, although the difference from the 2-EIA algorithm was not statistically significant (P=0.72). When all 1,300 control subjects from both study phases were considered together, the 2-EIA algorithm's specificity was 99.5%, equal to that of standard 2-tiered testing, but superior to the C6 ETA alone (specificity 98.4%, P=0.01).

Positive Predictive Value of the Various Approaches.

The positive predictive value (PPV) of the standard 2-tiered algorithm, the 2-EIA algorithm, and the C6 EIA as a stand-alone test, were calculated using the overall sensitivity and specificity values reported in Table 4. For disease prevalence 1.7% was used, which was the approximate prevalence of LD among those tested for the infection at our Boston hospital (4520 samples submitted for testing in 1 year; 78 LD patients identified by medical record review). The standard 2-tiered algorithm's PPV was 66% compared with 70% for the 2-EIA algorithm and 43% for the C6 EIA alone (Table 5). Thus, the small reduction in specificity of the C6 EIA, compared with 2-tiered testing, resulted in a marked reduction in the predictive value of a positive test result.

### DISCUSSION

A 2-tiered approach to serologic testing for LD was evaluated using 2 EIAs, the first a polyvalent WCS EIA and the second a VlsE C6 EIA. Compared with standard 2-tiered testing, the C6 EIA provided greater sensitivity in early infection, but less specificity. In contrast, the 2-EIA approach provided sensitivity that was very close to the C6 EIA, but maintained the specificity of standard 2-tiered testing without the technical complexity or subjective interpretation of Western blotting.

The 2-EIA approach was evaluated using 2 separate sera sets, each with different strengths. In phase 1, the 2-EIA algorithm was tested using sera obtained in a prospective research study. The patients in this study were well characterized, including culture confirmation for the EM patients. Therefore, there is a high degree of certainty that they were correctly classified. In phase 2, the 2-EIA system was evaluated using routine sera submitted to a clinical laboratory to simulate the “real world” situation. In both phases of the study, the 2-EIA algorithm performed better in early LD (stages 1 and 2) than standard 2-tiered testing, whereas the algorithms were equally sensitive in late infection (stage 3). Consistent with earlier reports (Bacon, et al., J. Infect. Dis. 187(8):1187-99 (2003); Mogilyansky, et al., Clin. Diagn. Lab. Immunol. 11(5):924-9 (2004); Steere, et al., Clin. Infect. Dis. 47(2):188-95 (2008)), the improvement in sensitivity in stage 1 resulted from use of a more sensitive C6 EIA as the second-tier assay rather than Western blotting.

In stage 2 LD, the improvement in sensitivity was attributable to an important difference in interpretive criteria between the 2 algorithms. According to the CDC criteria, a positive second-tier IgM Western blot can be used to support the diagnosis only in persons with illness ≦1 month's duration (Recommendations for test performance and interpretation from the Second National Conference on Serologic Diagnosis of Lyme Disease, Morb. Mortal Wkly Rep. 44(31):590-1 (1995)). This caveat was included to reduce false-positive results, since patients were expected to have positive IgG antibody responses after 1 month of illness. Although patients with stage 2 LD often have both IgM and IgG reactivity with early antigens, such as the 23- 39-, and 41-kD spirochetal proteins, it may take up to 2 months before they have IgG responses against 5 antigens as required for a positive IgG blot (Branda, et al., Clin. Infect. Dis. 50(1):20-6 (2010)). In the current study, 7 of 26 stage 2 LD patients had only positive IgM responses 5-8 weeks after disease onset, and therefore, they were classified as seronegative by standard 2-tiered testing, reducing the standard algorithm's clinical sensitivity. In contrast, the proposed 2-EIA algorithm is meant to be applied to any patient, regardless of duration of illness. Not only does this modification improve sensitivity in stage 2 LD compared with standard 2-tiered testing, it obviates the need to pinpoint the exact duration of illness, which can be difficult.

Certainly, there is a downside to the 2-EIA algorithm compared with Western blotting. With blots, one learns the spirochetal antigens against which the patient's antibody response is directed, and the response expands over time. This gives information about the duration of illness, and in cases in which the diagnosis is difficult, this information may be helpful. Thus, there is still a need for Western blotting in the armamentarium of serologic tests for Lyme disease. However, in routine cases, this level of detail is often superfluous and prone to misinterpretation.

Does the 2-EIA approach give enough additional information to justify the added labor and expense compared with use of the C6 EIA alone? Although the 2-EIA algorithm was slightly less sensitive in stage 1 LD than the C6 EIA alone (53% versus 56%, P=0.69), serologic testing is not recommended for stage 1 disease (Tugwell, et al., Ann. Intern. Med. 127(12):1109-23 (1997)), and the 2 approaches were equally sensitive in stages 2 and 3. This small difference in sensitivity was off-set by a significant difference in specificity, which favored the 2-EIA algorithm over the C6 EIA alone (99.5% versus 98.4%; P=0.01). This difference in specificity translates into large differences in PPV (70% versus 43% in the present study) because the prevalence of LD in tested populations is usually low, even in endemic areas. Furthermore, reducing specificity by 1 percentage point, as was shown here, changes the false-positive rate from 0.5% to 1.6%. In the U.S., where at least 3.4 million LD tests are done annually (Porwancher, et al., Clin. Vaccine Immunol. 18(5):851-9 (2011)), this difference would lead to an additional 37,000 false-positive results per year. Thus, the number of false-positive results would exceed the reported incidence of LD in the U.S. (>35,000 cases annually) (Centers for Disease Control and Prevention (CDC), Division of Vector-borne Infectious Diseases (DVBID), Reported cases of Lyme disease by year, United States, 1995-2009. http://www.cdc.gov/ncidod/dvbid/lyme/resources/Casenumbers-graph1995_{—}2009_b.pdf. Accessed 17 Feb. 2011). This finding, in our opinion, is the reason to retain a 2-test approach.

One could also consider variations of the 2-EIA strategy. One could reverse the order of the 2 tests, starting with the C6 EIA and in those with a positive or equivocal result, follow with a WCS EIA. This would increase the number of C6 tests while reducing the number of WCS EIA tests. However, because the C6 test is more expensive and may be more prone to quality control failures, the order of performing the WCS EIA first is probably preferable. Alternately, one could do both EIAs at the same time, rather than in step-wise fashion. Such an approach could be adapted to a simpler format, such as a lateral flow immunochromatographic test strip, potentially allowing for sensitive and specific diagnosis of LD at the point of care.

The present study had several limitations. First, testing was done at different times using frozen samples. However, all specimens were stored at −80° C., and freeze-thaw cycles were minimized. Secondly, samples were not tested from control subjects with other tick-borne infections, and further study is warranted. Third, control sera from the MGH blood bank were tested using a different WCS EIA than the other study specimens. However, both WCS EIAs use the same antigen target (B. burgdorferi strain B31), both are FDA-cleared and performed similarly in FDA trials.

In summary, a 2-tiered antibody test for Lyme disease consisting of a WCS ETA followed by a VlsE C6 EIA performs as well as, or better than, the standard 2-tiered approach. The 2-EIA algorithm provided sensitivity comparable to that of the C6-peptide EIA alone, but maintained equivalent specificity compared with standard 2-tiered testing. Thus, for routine testing, the standard 2-tiered algorithm could be replaced by 2 EIAs.

TABLE 1
Serologic responses in well-characterized Lyme disease study patients.

| | Number positive (%) | | | | | | |
|---|---|---|---|---|---|---|---|
| | | | | | | Standard 2- | |
| | | | | | IgM and/or | tiered | 2-EIA |
| Patients with Lyme disease (N = 91) | WCS EIA^{a} | C6 EIA^{a} | IgM WB^{b,c} | IgG WB^{c} | IgG WB^{b,c} | algorithm^{d} | algorithm^{e} |
| Stage 1: Erythema migrans (N = 63) | | | | | | | |
| Active phase | 33 (52) | 27 (43) | 15 (24) | 3 (5) | 18 (29) | 17 (27) | 23 (37) |
| Convalescent phase | 59 (94) | 58 (92) | 31 (49) | 5 (8) | 36 (57) | 36 (57) | 56 (89) |
| Stage 2: Acute neuritis or carditis (N = 10) | 10 (100) | 10 (100) | 9 (90) | 3 (30) | 10 (100) | 4 (40) | 10 (100) |
| Stage 3: Arthritis or late neuritis (N = 18) | 18 (100) | 18 (100) | 2 (11) | 18 (100) | 18 (100) | 18 (100) | 18 (100) |

WCS, whole-cell sonicate; EIA, enzyme immunoassay; WB, Western blot.
^{a}For the EIA results, the numbers represent serum samples with either positive or equivocal results.
^{b}In these columns, positive immunoglobulin M (IgM) Western blot results were reported regardless of the duration of illness prior to specimen collection.
^{c}All phase 1 sera were analyzed by Western blot regardless of whether the first-step WCS EIA was positive or negative.
^{d}In this column, IgM criteria were applied only when the duration of illness prior to specimen collection was ≦1 month. The numbers represent serum samples that were positive or equivocal by WCS EIA and positive by either IgM or IgG WB analysis.
^{e}In this column, the numbers represent serum samples that were positive or equivocal by whole-cell sonicate (WCS) EIA and positive or equivocal by C6-peptide EIA.

TABLE 2
Serologic responses in Lyme disease patients whose sera were submitted to
the Clinical Microbiology Laboratory at Massachusetts General Hospital

| | Number positive (%) | | | | | | |
|---|---|---|---|---|---|---|---|
| | | | | | | Standard 2- | |
| | | | | | IgM and/or | tiered | 2-EIA |
| Patients with Lyme disease (N = 78) | WCS EIA^{a} | C6 EIA^{a} | IgM WB^{b} | IgG WB | IgG WB^{b} | algorithm^{c} | algorithm^{d} |
| Stage 1: Erythema migrans (N = 51) | 51 (100)^{e} | 37 (73) | 31 (61) | 5 (10) | 32 (63) | 31 (61) | 37 (73) |
| Stage 2: Acute neuritis or carditis (N = 16) | 16 (100)^{e} | 16 (100) | 15 (94) | 3 (19) | 16 (100) | 15 (94) | 16 (100) |
| Stage 3: Arthritis (N = 11) | 11 (100)^{e} | 11 (100) | 5 (45) | 11 (100) | 11 (100) | 11 (100) | 11 (100) |

WCS, whole-cell sonicate; EIA, enzyme immunoassay; WB, Western blot.
^{a}For the enzyme immunoassay (EIA) results, the numbers represent serum samples with either positive or equivocal results.
^{b}In these columns, positive immunoglobulin M (IgM) Western blot results were reported regardless of the duration of illness prior to specimen collection.
^{c}In this column, IgM criteria were applied only when the duration of illness prior to specimen collection was ≦1 month.
^{d}In this column, the numbers represent serum samples that were positive or equivocal by whole-cell sonicate (WCS) EIA and positive or equivocal by C6-peptide EIA.
^{e}Patients with LD were identified by reviewing the medical record of patients with a positive first-step WCS EIA; therefore, all sera from LD patients were, by definition, positive by WCS EIA.

TABLE 3
Serologic responses in symptomatic or healthy control subjects

| | Number positive (%) | | | | | | |
|---|---|---|---|---|---|---|---|
| | | | | | | Standard 2- | |
| | | | | | IgM and/or | tiered | 2-EIA |
| | WCS EIA^{a} | C6 EIA^{a} | IgM WB^{b} | IgG WB^{b} | IgG WB^{b} | algorithm | algorithm^{c} |
| Symptomatic control subjects (N = 54)^{d} | 12 (22) | 1 (2) | 0 (0) | 0 (0) | 0 (0) | 0 (0) | 0 (0) |
| Healthy control subjects (N = 1246) | | | | | | | |
| CT or RI field sites (N = 66) | 4 (6) | 4 (6) | 0 (0) | 1 (2) | 1 (2) | 1 (2) | 1 (2) |
| Boston blood donors (N = 1080) | 16 (1) | 16 (1) | 4 (0) | 2 (0) | 6 (1) | 6 (1) | 6 (1) |
| New Zealand blood donors (N = 100) | 3 (3) | 0 (0) | 0 (0) | 0 (0) | 0 (0) | 0 (0) | 0 (0) |

WCS, whole-cell sonicate; EIA, enzyme immunoassay; WB, Western blot.
^{a}For the enzyme immunoassay (EIA) results, the numbers represent serum samples with either positive or equivocal results.
^{b}All sera from symptomatic control subjects, and healthy control subjects from CT, RI or New Zealand were analyzed by Western blot regardless of whether the first-step WCS EIA was positive or negative. In contrast, sera from healthy control subjects in Boston were only tested by Western blot if the first-step WCS EIA was positive or equivocal.
^{c}In this column, the numbers represent serum samples that were positive or equivocal by whole-cell sonicate (WCS) EIA and positive or equivocal by C6-peptide EIA.
^{d}Includes 25 patients with chronic fatigue syndrome or fibromyalgia, 14 with rheumatic diseases (such as rheumatoid arthritis or psoriatic arthritis), 9 with neurologic illnesses (including multiple sclerosis), 5 with other infections (including 2 with parvovirus B19 infection, 1 with hepatitis C infection, and 2 with presumed viral infection), and 1 with T cell lymphoma.

TABLE 4
Overall performance of the proposed 2-EIA algorithm

| | Standard 2-tiered | | | | | | | | | | |
|---|---|---|---|---|---|---|---|---|---|---|---|
| | algorithm | | | 2-EIA algorithm | | | | C6 EIA alone | | | |
| | | Sens | Spec | | Sens | Spec | | | Sens | Spec | |
| Patients with Active Lyme Disease | No.+ | (%) | (%) | No.+ | (%) | (%) | P value^{a} | No.+ | (%) | (%) | P value^{a} |
| Early Disease (N = 140) | 67 | 48 | — | 86 | 61 | — | 0.03 | 90 | 64 | — | 0.008 |
| Stage 1: Erythema migrans (N = 114) | 48 | 42 | — | 60 | 53 | — | 0.14 | 64 | 56 | — | 0.05 |
| Stage 2: Acute neuritis or carditis (N = 26) | 19 | 73 | — | 26 | 100 | — | 0.06 | 26 | 100 | — | 0.06 |
| Late Disease (N = 29) | | | | | | | | | | | |
| Stage 3: Arthritis or late neuritis (N = 29) | 29 | 100 | — | 29 | 100 | — | 1.0 | 29 | 100 | — | 1.0 |
| All Patients (N = 169) | 96 | 57 | — | 115 | 68 | — | 0.04 | 119 | 70 | — | 0.01 |
| Control Subjects (N = 1300) | 7 | — | 99.5 | 7 | — | 99.5 | 1.0 | 21 | — | 98.4 | 0.01 |

EIA, enzyme immunoassay; No., number; Sens, sensitivity; Spec, specificity
^{a}The P values pertain to the comparison with the standard 2-tiered algorithm

TABLE 5
Positive predictive value of the 3 algorithms.

| | C6 EIA | Standard 2-tiered | 2-EIA |
|---|---|---|---|
| | alone | algorithm | algorithm |
| PPV (%) if LD | 18 | 36 | 41 |
| prevalence is 0.5% | | | |
| PPV (%) if LD | 31 | 55 | 58 |
| prevalence is 1.0% | | | |
| PPV (%) if LD | 40 | 64 | 67 |
| prevalence is 1.5% | | | |
| PPV (%) if LD | 43 | 66 | 70 |
| prevalence is 1.7%^{a} | | | |
| PPV (%) if LD | 47 | 70 | 74 |
| prevalence is 2.0% | | | |

EIA, enzyme immunoassay; PPV, positive predictive value.
^{a}The numbers in this row, which are highlighted in bold font, indicate the PPV values calculated using the prevalence of LD among those tested at our hospital (1.7%), as determined in phase 2 of this study. The other PPV values were calculated using hypothetical prevalence rates, for comparison.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1-53. (canceled)

54. A method for diagnostic evaluation of a subject for infection by a bacterium belonging to the Borrelia burgdorferi sensu lato complex, comprising:
a) obtaining a test biological sample from said subject, wherein said test biological sample is in the form of a fluid;
b) assaying said test biological sample for antibodies recognizing a whole cell lysate of said bacterium;
c) assaying said test biological sample for antibodies that specifically recognize epitopes of the variable major protein-like sequence, expressed (VlsE) lipoprotein of said bacterium.

55. The method of claim 54 wherein said method is performed on a test biological sample from a human subject exhibiting symptoms that may be attributable to Lyme disease and said human subject is treated as infected by said bacterium if the assay of step b) indicates the presence of antibodies that recognize said whole cell lysate and the assay of step c) indicates the presence of antibodies that recognize said VlsE lipoprotein of said bacterium.

56. The method of claim 54, wherein said test biological sample is assayed in step b) and step c) by lateral flow immunochromatography.

57. The method of claim 54, wherein said bacterium is of a species selected from the group consisting of: Borrelia burgdorferi sensu stricto; Borrelia afzelii; and Borrelia garinii.

58. The method of claim 54, wherein, in step c), antibodies that specifically recognize the VlsE lipoprotein are assayed for using C6 peptide.

59. The method of claim 54, wherein:
a) said subject is a human and said test sample is blood, plasma or serum;
b) said test biological sample is assayed in step b) and step c) using lateral flow immunochromatography.

60. The method of claim 59, wherein said bacterium is of a species selected from the group consisting of: Borrelia afzelii; Borrelia garinii; and Borrelia burgdorferi sensu stricto.

61. A method of diagnostically evaluating a subject for infection by a bacterium belonging to the Borrelia burgdorferi sensu lato complex, comprising:
a) obtaining a test biological sample from said subject wherein said test biological sample is in the form of a fluid;
b) assaying said test biological sample for antibodies recognizing a whole cell lysate of said bacterium and/or for antibodies recognizing a specific antigen characteristic of said bacterium;
c) assaying said test biological sample for at least one antigen characteristic of said bacterium.

62. The method of claim 61 wherein said method is performed on a test biological sample from a human subject exhibiting symptoms that may be attributable to Lyme disease and said human subject is treated as infected by said bacterium if the assay of step b) indicates the presence of antibodies that recognize said whole cell lysate and/or the presence of antibodies that recognize said specific antigen and the assay of step c) indicates the presence of at least one antigen characteristic of said bacterium.

63. The method of claim 61, wherein, in step b) said test biological sample is assayed for antibodies recognizing a whole cell lysate of said bacterium and said antigen in step c) is selected from the group consisting of one or more of: VlsE; OspC; OspA; OspB; flagellar proteins; BmpA; or peptides derived from these proteins.

64. The method of claim 61, wherein said test biological sample is assayed in step b) and step c) using lateral flow immunochromatography.

65. The method of claim 61, wherein said bacterium is of a species selected from the group consisting of: Borrelia burgdorferi; Borrelia afzelii; and Borrelia garinii.

66. The method of claim 61, wherein said test biological sample is blood, plasma or serum.

67. A device for lateral flow immunochromatography comprising a surface, with the following components immobilized on said surface:
a) at least one antigen characteristic of a bacterium of the genus Borrelia; and/or
b) antibodies recognizing one or more antigens characteristic of the Borrelia burgdorferi sensu lato complex.

68. The device of claim 67, wherein the antigen of step a) is selected from the group consisting of: recombinant or purified VlsE, synthetic C6 peptide; recombinant or purified outer surface protein C (OspC); a synthetic peptide comprising the C-terminal 10-amino-acid residues of OspC (pepC 10); and the antibodies of step b) are directed against one or more of the following antigens: VlsE; OspC; OspA; OspB; flagellar proteins; BmpA; or peptides derived from these proteins.

69. The device of claim 67, wherein said device comprises both an antigen characteristic of a bacterium of the Borrelia burgdorferi sensu lato complex and an antibody recognizing an antigen characteristic of a bacterium of the Borrelia burgdorferi sensu lato complex.

70. The device of claim 67, wherein said surface comprises an absorbent material.

71. The device of claim 67, wherein said bacterium is of the species Borrelia burgdorferi sensu stricto.

72. The device of claim 67, wherein said bacterium is of the species Borrelia afzelii.

73. The device of claim 67, wherein said bacterium is of the species Borrelia garinii.
